(19) United States
(12) Patent Application Publication
(10) Pub. No.: US 2006/0147511 A1
(43) Pub. Date: Jul. 6, 2006

(54) Liposomal glucocorticoids

(76) Inventors: Steffen Panzner, Halle (DE); Rolf Brauer, Apolda (DE); Raimund W Kinne, Jena (DE); Una Rauchhaus, Halle (DE)

Correspondence Address: STEPHAN A. PENDORF, P.A., PENDORF & CUTLIFF, 5111 MEMORIAL HIGHWAY, TAMPA, FL, 33634, US

(21) Appl. No.: 10/536,200

(22) PCT Filed: Nov. 24, 2003
(86) PCT No.: PCT/DE03/03893

§ 371 Date: Nov. 28, 2005

(30) Foreign Application Priority Data
Nov. 24, 2002 (DE) 102 55 106.5

(51) Int. Cl. A61K 9/127; A61K 31/573

(52) U.S. Cl. 424/450; 514/179

(57) ABSTRACT

The invention relates to a liposomal formulation comprising water-soluble glucocorticoids, to the use of said formulation in the treatment of inflammatory diseases, and to a kit comprising said liposomal formulation, e.g. in the form of a pharmaceutical agent.

### BACKGROUND OF THE INVENTION

1. Field of the invention

The invention relates to a liposomal formulation comprising water-soluble glucocorticoids, to the use of said formulation in the treatment of inflammatory diseases, and to a kit comprising said liposomal formulation, e.g. in the form of a pharmaceutical agent.

2. Related Art of the Invention

Glucocorticoids are a class of substances which have been employed in the treatment of inflammatory diseases for a long time. Incorporation of active substances from this class in liposomal formulations is well-known in principle and has been described repeatedly. Formulation of a non-modified drug form is difficult because material enclosed in liposomes will be re-liberated within a few hours. A brief discussion of well-known strategies including examples will be given below:

Lipophilic derivatization for incorporation in the liposomal membrane: DE 27 120 30 (ICI 1977) is an early example that lipophilic-substituted glucocorticoids, e.g. dexamethasone palmitate, can be stably entrapped in liposomes (see also: Mizu-shima et al. 1982, Yokoyama et al. 1985, Bonanomi et al. 1987). What is disadvantageous in these formulations is the exchange of the hydrophobic active substance derivative with plasma components and low in vivo stability of the formulations.

PEG liposomes: One improvement with respect to the above has been disclosed in WO 94/07466 (Liposome Technology). By using lipids modified with PEG (polyethylene glycol), it is possible to increase the in vivo stability of the formulation and, in particular, extend the circulation time of the liposomes. In principle, this improvement is directed to reduce non-specific loss of incorporated liposomes in the reticuloendothelial system, thereby enabling or improving specific extravasation of liposomes at centers of inflammation.

Passive enclosure of water-soluble glucocorticoids in liposomes is possible. Derivatives such as phosphate esters, acetonide phosphates and/or succinates are suitable for this purpose. More recent developments, such as EP 1 046 394 (Terumo Corp) and WO 02/45688 (Gerrit Storm), have taken this route where water-soluble glucocorticoids are enclosed in PEG-modified liposomes (see also: Storm & Richmond 2001, Metselaar et al. 2002, Hussein et al. 2002).

Cyclodextrin complexes with glucocorticoids represent another option of creating a water-soluble derivative which can be incorporated in liposomes in the usual way (Loftsson & Stefansson 2002, Yano et al. 2002, WO 95/15746 (Gregoriadis)).

Furthermore, WO 99/15150, WO 99/55314 and EP 1 190 707 disclose liposomes containing water-soluble glucocorticoids.

The prior art involves the disadvantage that the improvements in circulation behavior being achieved essentially can only be obtained when the formulation is administered for the first time. Where PEG-modified liposomes are administered repeatedly, formation of antibodies can be detected. When used repeatedly, binding thereof will result in a decreased circulation half-life and is undesirable in pharmacological terms (Dams et al. 2000).

### SUMMARY OF THE INVENTION

The object of the invention is to provide liposomal formulations of glucocorticoids, which avoid the above-mentioned drawbacks and exhibit an advantageous effect compared to the free active substance.

The object of the invention is accomplished by providing a liposomal formulation which has water-soluble glucocorticoids in an interior aqueous phase, does not comprise any amphiphilic-modified polyethylene glycol and is stable for hours in animal or human blood serum.

In the meaning of the invention, “stable for hours” means that the formulation will be stable in serum for at least one, preferably two, more preferably three to four hours, and especially preferably for five to 24 hours.

Surprisingly, unlike well-known structures, the liposomal formulations of the invention are stable in serum of organisms for a long period of time. According to the present patent application, the feature of stability therefore should not be understood to be an object to a person skilled in the art, but is merely used for clarification relating to the fact that the described liposomal formulations of the invention exhibit said feature. The liposomal formulations or liposomes of the invention are essentially free of conjugated polymers and, in particular, free of PEG, PEG-phosphatidyl ethanolamine, PEG-phosphatidyl glycerol, PEG-phosphatidyl serine and/or PEG-cholesterol or other amphiphilic-modified polyethylene glycols.

Surprisingly, liposomal preparations of glucocorticoids produced without addition of PEG lipids were found to be sufficiently stable in systemic application, having a highly advantageous effect when compared to free glucocorticoids, so that an improved therapeutic effect can be achieved with lower dosage. Accordingly, the liposomal formulations, liposomal preparations or liposomes of the invention can be used in various fields of diagnosis, prophylaxis and therapy. In a preferred fashion, the structures according to the invention can be employed in the therapy of inflammatory diseases. A preferred selection of indications of glucocorticoids is:
severe allergic and anaphylactic reactions,
anaphylactic shock,
status asthmaticus, severe conditions in bronchial asthma,
brain oedema, craniocerebral trauma, brain abscess, bacterial meningitis,
parenteral initial treatment of acute severe dermal diseases (erythrodermia, pemphigus vulgaris, acute eczemas),
severe infectious diseases, adrenocortical insufficiency,
idiopathic retroperitoneal fibrosis, Addison's disease,
adrenogenital syndrome, chronic aggressive hepatitis,
lymphogranulomatosis, differential diagnosis of Cushing's disease,
primary chronic polyarthritis (rheumatoid arthritis), chronic polyarthritis, juvenile arthritides (Still syndrome), acute rheumatic carditis, arthroses, activated arthrosis, acute form of periarthropathia humeroscapularis, non-bacterial tendovaginitis and bursitis,
periarthropathies, insertion tendopathies, psoriatic arthritis,
ankylosing spondylitis (Bechterew's disease),
spondylo-arthroses with synovitis (activated arthrosis), spondyloses,
giant cell arteritis, Wegener's disease, Churg-StrauB syndrome, microscopic polyangiitis,
non-infectious keratoconjunctivitis, scleritis, iridocyclitis, uveitis,
collagenoses (systemic lupus erythematosus (especially nephritis), panarteritis nodosa, dermatomyositis, primary Sjogren syndrome),
acute attacks of Crohn's disease, severe attacks of ulcerative colitis (e.g. toxic colon),
multiple myeloma,
PFAFA syndrome (periodic fever in children),
idiopathic retroperitoneal fibrosis (Ormond's disease),
multiple sclerosis, acute myasthenia gravis crisis, angioneurotic oedemas (e.g. Quincke syndrome), and/or
acute alveolitis.

It goes without saying that different water-soluble glucocorticoids can be used. In particular, the use of the glucocorticoids being employed depends on the specific routes of application. Preferred are systemically applied glucocorticoids: betamethasone, betamethasone dihydrogen phosphate disodium, budesonide, cloprednol, cortisone, cortisone acetate, deflazacort, dexamethasone, dexamethasone dihydrogen phosphate disodium, fluocortolone, hydrocortisone, methylprednisolone, prednisolone, prednisone, prednylidene, rimexolone and/or triamcinolone.

Those preferably applied on the dermal route are: alclometasone, amcinonide, betamethasone, clobetasol, clobetasone, clocortolone, desonide, desoxymethasone, dexamethasone, diflorasone, diflucortolone, fludroxycortide, flumethasone, flucinolone, flucinonide, fluocortin, fluocortolone, fluorometholone, fluprednidene, fluticasone, halcinonide, halomethasone, hydrocortisone, methylprednisolone, mometasone, prednicarbate, prednisolone and/or triamcinolone.

In the context with this invention, the systemically applied glucocorticoids selected from those listed above are preferably employed in a water-soluble form. Water-soluble forms known to those skilled in the art are salts and esters of the active substances mentioned above such as, in particular, phosphate esters, sulfate esters or dicarboxylic esters or addition salts or glycosides of the active substances.

In a preferred fashion, mono- or di-salts are used, more preferably sodium or potassium salts of glucocorticoid phosphates, acetonide phosphates or succinates.

Particularly preferred compounds from this group are betamethasone dihydrogen phosphate disodium, dexamethasone phosphate, dexamethasone dihydrogen phosphate disodium, prednisolone phosphate, methylprednisolone phosphate, prednisolone succinate, methylprednisolone succinate, betamethasone phosphate, desonide phosphate, hydrocortisone phosphate, hydrocortisone succinate, prednisol-atma hydrochloride, prednisone phosphate and/or triamcinolone acetonide phosphate.

Highly preferred compounds from this group are dexamethasone phosphate, dexamethasone dihydrogen phosphate disodium and/or triamcinolone acetonide phosphate.

The preferred compounds according to the invention are particularly suitable for use against inflammatory diseases. The use of the liposomal formulations according to the invention results in advantages and improvements compared to well-known liposomal formulations, especially in increased reliability and quality and greater effectiveness in the prophylaxis and therapy of the diseases mentioned above. As a consequence, the liposomes according to the invention represent an enlargement to the drug resources, increasing the prophylactic and therapeutic potential of treating various diseases. As the liposomal formulations according to the invention exhibit improved activity compared to the free active substance, the latter can be used at lower doses, resulting in a reduced spectrum of side effects—an advantage in the therapy using glucocorticoids which should not be underestimated. Furthermore, material, cost, chemicals and pharmaceutical agents difficult to obtain are saved as a result of such dose reduction. Hence, the structures according to the invention satisfy an urgent need that has been unresolved for a long time.

Those skilled in the art will be familiar with liposomes that must be employed in order to achieve highest possible serum stability and prevent formation of aggregates. In a particularly preferred fashion, liposomes having a size between 150 nm and 500 nm are used.

Liposomes of this type are well-known to those skilled in the art, including liposomes constituted of saturated phospholipids and cholesterol. In a preferred embodiment of the teaching according to the invention, the following lipids are used to build up the liposomes: dipalmitoylphosphatidyl choline, distearoylphosphatidyl choline, dipalmitoylphosphatidyl glycerol, distearoylphosphatidyl glycerol, dipalmitoylphosphatidyl serine, distearoylphosphatidyl serine, cholesterol.

Preferred among mixtures of these substances are those having a percentage of negative charge carriers of no more than 20 mole-%. Also preferred are mixtures wherein the percentage of negative charge carriers is between 5 and 15 mole-%. Phosphatidyl glycerols and phosphatidyl serines are negative charge carriers in such membranes.

The lipid mixtures preferred according to the invention preferably include those containing either no cholesterol or between 35 and 50 mole-% cholesterol. Particularly preferred are liposomes containing between 35 and 45 mole-% cholesterol.

pH-sensitive liposomes are also well-known, including especially cholesterol hemisuccinate (CHEMS) because the carboxyl group thereof is discharged more and more below pH 5. This lipid is used with advantage in combination with phosphatidyl ethanolamine. Such liposomes, with a content of 35 to 50 mole-% CHEMS and 65 to 50 mole-% phosphatidyl ethanolamine, are stable at neutral pH value, undergoing decomposition at a pH value below 5 which, for example, is attained during endosomal uptake. Instead of CHEMS, it is also possible to use another pH-sensitive lipid, e.g. phosphatidyl serine.

In a particularly preferred embodiment of the invention, amphoteric liposomes are used. One special feature of these liposomes is that their surface charge changes with the pH value of the environment from cationic in the acidic to anionic in the basic pH range.

WO 02/066012 describes pH-sensitive amphoteric liposomes, the amphoteric behavior of which is determined by the simultaneous presence of cationic and anionic charge carriers in the membrane and can be varied by suitable selection and ratio of the components.

As negative charge carriers of amphoteric liposomes according to WO 02/066012, natural lipids are preferably employed, especially dipalmitoylphosphatidyl glycerol, distearoylphosphatidyl glycerol, dipalmitoylphosphatidyl serine, distearoylphosphatidyl serine, but also lipids produced by synthesis, such as cholesterol hemisuccinate—or CHEMS—diacyl-glycerol hemisuccinates.

As cationic charge carriers of amphoteric liposomes according to WO 02/066012, lipids can be used, preferably:
DC-Chol 3-β-[N-(N′,N′-dimethylethane)carbamoyl]cholesterol,
TC-Chol 3-β-[N-(N′,N′,N′-trimethylaminoethane)carbamoyl]-cholesterol,
BGSC Bis-guanidinium-spermidine-cholesterol,
BGTC Bis-guanidinium-tren-cholesterol,
DOTAP (1,2-dioleoyloxypropyl)-N,N,N-trimethylammonium chloride,
DOSPER (1,3-dioleoyloxy-2-(6-carboxyspermyl)propylamide),
DOTMA (1,2-dioleyloxypropyl)-N,N,N-trimethylammonium chloride (Lipofectin®),
DORIE (1,2-dioleyloxypropyl)-3-dimethylhydroxyethylammonium bromide,
DOSC (1,2-dioleoyl-3-succinyl-sn-glycero-choline ester),
DOGSDSO (1,2-dioleoyl-sn-glycero-3-succinyl-2-hydroxyethyl disulfide ornithine),
DDAB dimethyldioctadecylammonium bromide,
DOGS ((C18)₂GlySper3⁺) N,N-dioctadecylamido-glycylspermine (Transfectam®),
(C18)₂Gly⁺ N,N-dioctadecylamidoglycine,
CTAB cetyltrimethylammonium bromide,
CPyC cetylpyridinium chloride,
DOEPC 1,2-dioleoyl-sn-glycero-3-ethylphosphocholine or other O-alkylphosphatidyl cholines or ethanolamines and/or
amides of lysine, arginine or ornithine and phosphatidyl ethanolamine.

Also, pH-sensitive positive charge carriers can be components of amphoteric liposomes according to WO 02/066012. It is preferred to use lipids, such as disclosed in WO 02/066490 and WO 03/070220, e.g. 4-(2-aminoethyl)morpholinocholesterol hemisuccinate and/or histaminylcholesterol hemisuccinate.

In a preferred embodiment of the teaching according to the invention, a lipid matrix including the following lipids is built up with neutral backbone lipids in order to construct the amphoteric liposomes:

dimyristoylphosphatidyl choline, dipalmitoylphosphatidyl choline, palmitoyloleoylphosphatidyl choline, distearoylphosphatidyl choline, cholesterol, as well as purified choline fractions of natural origin, such as soy phosphatidyl choline or egg phosphatidyl choline.

WO 02/066489 describes amphoteric liposomes, the amphoteric behavior of which is determined by amphoteric charge carriers in the membrane which are derived from cholesterol, such as Na-histidinylcholesterol hemisuccinate. WO 03/070735 describes components for other pH-sensitive liposomes of this type.

In a preferred fashion the liposomal formulations have advantageous mixing ratios. Among the mixtures of these substances, those having a percentage of negative charge carriers of no more than 40 mole-% are preferred. Particularly preferred are mixtures wherein the percentage of negative charge carriers is between 5 and 30 mole-%.

Among the mixtures of these substances, those having a percentage of positive charge carriers of no more than 40 mole-% are preferred. Particularly preferred are mixtures wherein the percentage of positive charge carriers is between 5 and 30 mole-%.

In backbone lipids, the molar percentage in the composition of the liposomal membrane is preferably 30 to 80%, more preferably 40 to 70%.

The lipid mixtures preferred according to the invention preferably include those containing either no cholesterol or between 35 and 50 mole-% cholesterol. Particularly preferred are liposomes containing between 35 and 45 mole-% cholesterol.

Where amphoteric lipids are used in the formation of the liposomes, the molar percentage is less than 40 mole-%, preferably 5 to 30 mole-%.

Such amphoteric liposomes are particularly preferred when carrying out the invention, because they are stable in serum and do not form any aggregates. The disclosures of the pertaining citations, i.e. WO 02/066012, WO 02/066490, WO 02/066489, WO 03/070220, WO 03/070735, are hereby incorporated in the disclosure of the present description.

Methods of producing liposomes containing active substances are well-known to those skilled in the art and can be inferred from standard textbooks.

In a particularly advantageous fashion, the lipids are dissolved in a pharmacologically acceptable alcohol such as ethanol, isopropanol or 1,2-propanediol, or DMSO as well, and diluted in the aqueous solution of the active substance with rapid mixing. When conducting this process in an appropriate manner, e.g. at 10 fold dilution of the organic phase in the aqueous phase, liposomes in the desired size range of between 150 and 500 nm will form. These liposomes do not require any additional modification by homogenization or extrusion. Non-entrapped active substance can be removed. Procedures such as gel filtration, centrifugation or dialysis, especially tangential flow dialysis, are suitable to this end.

The invention also relates to pharmaceutical agents comprising the liposomal formulations according to the invention. The liposomes of the invention are formulated into a pharmaceutical preparation. This includes the use and/or addition by mixing of adjuvants (stabilizers, antioxidants) ensuring good tolerability, pharmaceutical safety and production and storage under GMP conditions. For example, this includes the use of sterile salt, buffer and/or sugar solutions.

The compounds according to the invention are contacted with an organism in a therapeutic amount; in this event, the compounds of the invention are employed as pharmaceutical agents or drugs; accordingly, these terms can be used synonymously in the context with this invention. The expression “therapeutical amount” as used herein refers to an amount that prevents or improves symptoms of a disorder or of a responsive, pathologically physiological condition. In specific embodiments of the present invention the amount administered is sufficient to prevent or inhibit inflammation. As stated above, the invention therefore relates to pharmaceutical agents or drugs comprising the compounds of the invention, optionally together with pharmaceutical adjuvants.

The amount of compounds of the invention to be used in a healthy person in the event of prophylaxis or in a patient in the event of therapy is formulated and the dose established according to conventional medical practice, considering the disorder to be treated, the condition of each individual patient, the site of administration, the procedure of administration and other factors well-known to the attending physicians. Similarly, the dose of the administered compounds of the invention depends on the characteristics of the inflammation, on the in vivo half-life of the compounds of the invention in plasma, and on the concentration of the compounds of the invention in the formulation, and also on the route of administration, site and rate of dosage, clinical tolerance of each individual (human and animal), pathological affection of the patient and the like, as is well-known to physicians or other persons skilled in the art. It is also possible to employ varying dosages during a sequence of consecutive administrations.

For example, injections (intramuscular or subcutaneous or into blood vessels) are envisaged as a route of therapeutic administration of the compounds of the invention, e.g. encapsulated or carrier-bound compounds of the invention, although supply in the form of an aerosol, via catheters or surgical tubes is also applicable. Other preferred routes include commercially available nebulizers for liquid formulations and inhalation of lyophilized or aerolyzed compounds and suppositories for rectal or vaginal administration. The suitability of the selected parameters, e.g. dosage, regimen, selection of adjuvants and the like can be determined by taking serum aliquots from the patient, i.e. human or animal, and testing during the course of the applications. Alternatively or concomitantly, the amount of T cells or other cells of the immune system can be determined in a conventional manner so as to obtain an overall survey of the patient's inflammation. In addition, the clinical condition of the patient can be observed for the desired effect. As inflammations can be associated with other diseases, additional comonitoring of the latter is also possible.

In general, both aqueous formulations and dry compounds of the invention can be mixed with an excipient to provide a stabilizing effect prior to treatment e.g. with a solvent. An aqueous solution of a compound according to the invention can be an inventive compound in suspension or a solution. Other forms of preparation, presentation and application are well-known to those skilled in the art, e.g. as a gel, emulsion, brew-up formulation, drops, concentrate, syrup, bolus, aerosol, spray and/or inhalant. The treatment of inflammations comprises prophylaxis, prevention, diagnosis, attenuation, therapy, follow-up and/or aftercare.

The compound of the invention can be incorporated in a solution together with a pharmaceutically acceptable preservative. Examples of suitable preservatives of suspensions or solutions include phenol, benzyl alcohol, m-cresol, methylparaben, propylparaben, benzalkonium chloride and benzethonium chloride at such a concentration that a preserving effect occurs without adversely modifying the liposomal formulation. In general, the formulations of the compounds according to the invention may include components in amounts that will not adversely affect the production of stable forms, and in amounts suitable for effective, safe pharmaceutical administration. For example, other pharmaceutically acceptable excipients well-known to those skilled in the art may form part of the compounds or formulations according to the invention. For example, these include salts, various fillers, additional buffer agents, chelating agents, antioxidants, co-solvents and the like.

It is well-known to those skilled in the art that artificial or natural membranes of liposomes may have an immunestimulating effect, especially in those cases where the components are coupled to the surface of liposomes or entrapped inside the liposomes or simply mixed together with the liposomes. For example, such formulations of liposomes can be applied on the parenteral route together with the liposomes according to the invention; of course, it is also possible to modify the liposomes of the invention so as to have an immune-stimulating effect. Using well-known methods, e.g. a spray, such formulations can be applied nasally on the mucosa. In a preferred fashion, therapeutic treatment using a spray is suitable for treating inflammations in the ear-nose-throat region. The liposomal composition applied together with the composition according to the invention may comprise one or more additional pharmaceutical carriers selected from surface-active substances and absorption-promoting agents such as bile salts and derivatives thereof, fusidinic acid and derivatives thereof, oleic acid, lecithin, lysolecithins, etc., water-absorbing polymers such as glycofurol, polyvinylpyrrolidone, propylene glycol or polyacrylic acid, gelatin, cellulose and derivatives etc.; substances inhibiting enzymatic degradation, such as aprotinin etc.; organic solvents such as alcohols, e.g. ethanol, glycerol, benzyl alcohol etc.; or ethyl acetate etc.; hydrophobic agents such as vegetable oil, soybean oil, peanut oil, coconut oil, corn oil, olive oil, sunflower oil, “miglyols” or mixtures thereof, etc.; pH regulators such as nitric acid, phosphoric acid, acetic acid, citrates, etc.; preservatives and agents regulating the osmotic pressure, such as glycerol, sodium chloride, methyl para-oxybenzoate, benzoic acid, etc.; liposomes and/or emulsion formulations such as lecithins etc.; micro-encapsulated formulations; propellants such as butane.

In the meaning of the invention, the carriers which can be components of the drugs comprising the compounds of the invention can also be water-oil emulsions such as montanide, polylysine, polyarginine compounds, or others such as phosphate-buffered saline, water, sterile solutions and the like.

In addition to glucocorticoids, the pharmaceutical agent in the meaning of the invention may comprise, for example, an acceptable salt thereof or components thereof. For example, these can be salts of inorganic acids such as phosphoric acid or salts of organic acids. Furthermore, the salts can be free of carboxyl groups and derived from inorganic bases such as sodium, potassium, ammonium, calcium or iron hydroxides, or from organic bases such as isopropylamine, trimethylamine, 2-ethylaminoethanol, histidine and others. Examples of liquid carriers are sterile aqueous solutions including no further materials or active ingredients, and e.g. water, or those comprising a buffer such as sodium phosphate with a physiological pH or a physiological salt solution or both, such as phosphate-buffered sodium chloride solution. Other liquid carriers may comprise more than just one buffer salt, e.g. sodium and potassium chloride, HEPES, dextrose, propylene glycol or others. Liquid compositions of the pharmaceutical agents may additionally comprise a liquid phase, with water being excluded, however. Examples of such additional liquid phases are glycerol, vegetable oils, organic esters or water-oil emulsions. The pharmaceutical composition or pharmaceutical agent typically includes a content of at least 0.01 wt.-% of compounds according to the invention, relative to the overall pharmaceutical composition. The respective dose or dosage range for administering the pharmaceutical agent according to the invention is sufficiently high in order to achieve the desired prophylactic or therapeutic effect. In this context, the dose should not be selected in such a way that undesirable side effects would dominate. In general, the dose will vary with the patient's age, constitution, sex and, of course, depending on the severity of the disease. The individual dose can be adjusted both with reference to the primary disease and with reference to the occurrence of additional complications. Using well-known means and methods, the exact dose can be determined by a person skilled in the art, e.g. by determining the tumor growth as a function of dosage or as a function of the application regimen or pharmaceutical carrier and the like. Depending on the patient, the dosage can be selected individually. For example, a dose of pharmaceutical agent just tolerated by a patient can be such that the range thereof in plasma or locally in particular organs is from 0.1 to 10,000 μM, preferably between 1 and 100 μM, and this dose may relate to the pharmaceutical agent, the liposomal formulation or the glucocorticoids. Alternatively, the dose can be calculated relative to the body weight of the patient. Furthermore, however, it is also possible to determine the dose on the basis of particular organs rather than the whole patient. For example, this would be the case when placing the pharmaceutical agent according to the invention, e.g. in a biopolymer incorporated in the respective patient, near specific organs by means of surgery. Several biopolymers capable of liberating liposomes in a desirable manner are known to those skilled in the art. In this event, the therapeutic agent is administered as a solid, gel-like or liquid composition.

In another preferred embodiment of the invention, the carriers are selected from the group of fillers, diluents, binders, humectants, disintegrants, dissolution retarders, absorption enhancers, adsorbents and/or lubricants which are selected in such a way that the liposomal formulation of the invention will not be impaired.

The fillers and diluents are preferably starches, lactose, cane-sugar, glucose, mannitol and silica, the binder is preferably carboxymethylcellulose, alginate, gelatin, polyvinylpyrrolidone, the humectant is preferably glycerol, the disintegrant is preferably agar, calcium carbonate and sodium carbonate, the dissolution retarder is preferably paraffin, and the absorption enhancer is preferably a quaternary ammonium compound, the adsorbent is preferably kaolin and bentonite, and the lubricant is preferably talc, calcium and magnesium stearates, or mixture(s) of the materials mentioned above.

In another preferred embodiment of the invention the inventive compounds are prepared as gel, emulsion, brew-up formulation, drops, concentrate, syrup, bolus, aerosol, spray and/or inhalant and/or applied in this form.

The active substance(s), i.e., the compounds of the invention, optionally can be present in a micro-encapsulated form, together with one or more of the above-mentioned carrier substances.

In addition to the active substance(s), suppositories may include conventional water-soluble or water-insoluble carrier substances, e.g. polyethylene glycols, fats, e.g. cocoa fat and higher esters (for example, C₁₄ alcohol with C₁₆ fatty acid) or mixtures of such materials.

In addition to the active substance(s), ointments, pastes, creams and gels may include conventional carrier substances, e.g. animal and vegetable fats, waxes, paraffins, starch, tragacanth, cellulose derivatives, polyethylene glycols, silicones, bentonites, silica, talc and zinc oxide or mixtures of these materials.

Sprays may additionally include conventional propellants, e.g. chlorofluorohydrocarbons.

In addition to the active substance(s), solutions and emulsions may include conventional carrier substances such as solvents, solubilizers and emulsifiers, e.g. water, ethyl alcohol, isopropyl alcohol, ethyl carbonate, ethyl acetate, benzyl alcohol, benzyl benzoate, propylene glycol, 1,3-butylene glycol, dimethylformamide, oils, especially cotton seed oil, peanut oil, corn oil, olive oil, castor oil and sesame oil, glycerol, glycerol formal, tetrahydrofurfuryl alcohol, and fatty esters of sorbitans or mixtures of these materials. For parenteral application, the solutions and emulsions can also be present in sterile and blood-isotonic form.

In addition to the active substance(s), suspensions may include conventional carrier substances such as liquid diluents, e.g. example water, ethyl alcohol, propylene glycol, suspending agents, e.g. ethoxylated isostearyl alcohols, polyoxyethylenesorbitol and sorbitan esters, micro-crystalline cellulose, aluminum metahydroxide, bentonite, agar and tragacanth or mixtures of these materials.

The drugs can be present in the form of a sterile injectable preparation, e.g. as a sterile injectable aqueous or liposomal suspension. Such a suspension can be formulated by means of methods well-known in the art, using suitable dispersing or wetting agents and suspending agents. The sterile injectable preparation can also be a sterile injectable solution or suspension in a non-toxic parenterally tolerable diluent or solvent, e.g. a solution in 1,3-butanediol. Tolerable vehicles and solvents that can be used include mannitol, water, Ringer's solution and isotonic sodium chloride solution.

The formulated forms mentioned above may also contain colorants, preservatives, as well as odor- and taste-improving additives, e.g. sweeteners such as saccharine. Preferably, the compounds according to the invention should be present in the above-mentioned pharmaceutical preparations at a concentration of about 0.1 to 99.5, more preferably about 0.5 to 95 wt.-% of the overall mixture.

In addition to the compounds of the invention, the above-mentioned pharmaceutical preparations may include further pharmaceutical active substances. The production of the pharmaceutical preparations specified above proceeds in a usual manner according to well-known methods, e.g. by mixing the active substance(s) with the carrier substance(s).

The above-mentioned preparations can be applied orally, nasally, rectally, regionally, e.g. in joint regions or the like, parenterally (intravenous, intramuscular, subcutaneous routes), intracisternally, intravaginally, intraperitoneally, locally, dermally (powder, ointment, drops) in humans and animals and used in the therapy of inflammations in hollow areas and body cavities. For oral therapy, injection solutions, solutions and suspensions, gels, brew-up formulations, emulsions, ointments or drops are possible as suitable preparations. For local therapy, ophthalmic and dermatological formulations or solutions can be used. With animals, ingestion can be effected via feed or drinking water in suitable formulations. Furthermore, gels, tablets, sustained-release tablets, premixes, concentrates, boli, capsules, aerosols, sprays, inhalants can be used in humans and animals. Furthermore, the compounds of the invention can be incorporated in other carrier materials such as plastics (plastic chains for local therapy), collagen or bone cement.

The amount of active substance, i.e., the amount of a compound according to the invention, that is combined with the carrier materials to produce a single dosage form can be varied by a person skilled in the art depending on the host to be treated and on the particular type of administration. Once the condition of a host or patient has improved, the proportion of active compound in the preparation can be modified so as to obtain a maintenance dose. Depending on the symptoms, the dose or frequency of administration or both can subsequently be reduced to a level where the improved condition is retained. Once the symptoms have been alleviated to the desired level, the treatment should be stopped. However, patients may require an intermittent treatment on a long-term basis if any symptoms of the disease should recur. Accordingly, the proportion of the compounds, i.e. their concentration, in the overall mixture of the pharmaceutical preparation, as well as the composition or combination thereof, is variable and can be modified and adapted by a person of specialized knowledge in the art.

Those skilled in the art will be aware of the fact that the compounds of the invention can be contacted with an organism, preferably a human or an animal, on various routes. Furthermore, a person skilled in the art will also be familiar with the fact that the pharmaceutical agents in particular can be applied at varying dosages. Application should be effected in such a way that an inflammation is combatted as effectively as possible, or the onset of such a disease is prevented by a prophylactic administration. Concentration and type of application can be determined by a person skilled in the art using routine tests. Preferred applications of the compounds of the invention are oral application in the form of juice, drops, capsules or the like, rectal application in the form of suppositories, solutions and the like, parenteral application in the form of injections, infusions and solutions, inhalation of vapors and aerosols, and local application in the form of ointments, pads, dressings, lavages and the like. Contacting with the compounds according to the invention is preferably effected in a prophylactic or therapeutic fashion. In prophylactic administration, development of an inflammation is to be prevented at least in such a way that further inflammation is massively reduced or almost completely eliminated. In therapeutic contacting, a manifest inflammation of the patient is already existing, and the centers of inflammation already present in the body are to be inhibited. Other forms of application preferred for this purpose are e.g. subcutaneous, sublingual, intravenous, intramuscular, intraperitoneal and/or topical ones.

In addition to the above-specified concentrations during use of the compounds of the invention, the compounds in a preferred embodiment can be employed in a total amount of 0.005 to 500 mg/kg body weight per 24 hours, preferably 0.05 to 100 mg/kg body weight. Advantageously, this is a therapeutical quantity which is used to prevent or improve the symptoms of a disorder or of a responsive, pathologically physiological condition. The amount administered is sufficient to inhibit inflammation.

Obviously, the dose will depend on the age, health and weight of the recipient, degree of the disease, type of required simultaneous treatment, frequency of the treatment and type of the desired effects, and side-effects. The daily dose of 0.005 to 500 mg/kg body weight can be applied as a single dose or multiple doses in order to furnish the desired results. The dosage levels per day are applicable both in prophylaxis and treatment. In particular, pharmaceutical agents are typically used in about 1 to 10 administrations per day, or alternatively or additionally as a continuous infusion. However, they can also be administered at intervals of 2-10 days. Such administrations can be applied as a chronic or acute therapy. Of course, the amounts of active substance that are combined with the carrier materials to produce a single dosage form may vary depending on the host to be treated and on the particular type of administration. In a preferred fashion, the daily dose is distributed over 2 to 5 applications, with 1 to 2 tablets including an active substance content of 0.05 to 500 mg/kg body weight being administered in each application. Of course, it is also possible to select a higher content of active substance, e.g. up to a concentration of 5,000 mg/kg. For example, the capsules including the liposomes according to the invention can also be sustained-release capsules, in which case the number of applications per day is reduced to 1 to 3, or there may be an interval of several days between applications. The active substance content of sustained-release capsules can be from 3 to 3,000 mg.

If the active substance—as set forth above—is administered by injection, the host is preferably contacted with the compounds of the invention 1 to 10 times per day or by using continuous infusion, in which case amounts of from 0.04 to 4,000 mg per day are preferred. The preferred total amounts per day were found advantageous both in human and veterinary medicine. It may become necessary to deviate from the above-mentioned dosages, and this depends on the nature and body weight of the host to be treated, the type and severity of the disease, the type of preparation and application of the drug, and on the time period or interval during which the administration takes place. Thus, it may be preferred in some cases to contact the organism with less than the amounts mentioned above, while in other cases the amount of active substance specified above has to be surpassed. A person of specialized knowledge in the art can easily determine the optimum dosages required in each case and the type of application of the active substances.

In another particularly preferred embodiment of the invention the compounds of the invention are used in a single administration of from 0.01 to 80, especially from 0.3 to 40 mg/kg body weight. In the same way as the total amount per day, the amount of a single dose per application can be varied by a person of specialized knowledge in the art. The compounds used according to the invention can be administered with the above-mentioned single concentrations and preparations together with the feed or feed preparations or drinking water in veterinary medicine as well.

In another preferred embodiment of the invention the compounds according to the invention can be employed together with at least one other well-known pharmaceutical agent. That is to say, the compounds of the invention can be used in a prophylactic or therapeutic combination in connection with well-known drugs. Such combinations can be administered together, e.g. in an integrated pharmaceutical formulation, or separately, e.g. in the form of a combination of tablets, injection or other medications administered simultaneously or at different times, with the aim of achieving the desired prophylactic or therapeutic effect. These well-known agents can be agents which enhance the effect of the compounds according to the invention.

It goes without saying that the compounds of the invention, especially the pharmaceutical agents, can be used alone or together with other agents in a therapy, e.g. in a combination therapy, in the form of a region therapy.

Of course, the compounds of the invention can be used in combination with other well-known anti-inflammatory agents. Such agents are well-known to those skilled in the art. Accordingly, the compounds of the invention can be administered with any conventional agent, particularly other drugs available for use, either as a single drug or in a combination of drugs. They can be administered alone or in a combination with same.

The pharmaceutical composition can be present in substance or as an aqueous solution together with other materials such as preservatives, buffer substances, agents provided for osmolarity adjustment of the solution, and so forth.

The invention also relates to a kit and to the use thereof in medicine. In a preferred fashion, the compounds of the invention or the kit comprising same are used in a combination therapy, especially in the treatment of inflammations.

The liposomes according to the invention are used in systemic treatment of inflammatory and autoimmune diseases, using corticoids.

Preferred indications are autoimmune diseases. Inter alia, these include systemic vasculitides, lupus erythematosus, polymyositis, polymyalgia rheumatica, rheumatic fever, rheumatic rheumatoid arthritis, systemic sclerosis, reactive arthritides, neurodermatitis, psoriasis, Crohn's disease, ulcerative colitis, multiple sclerosis, bronchial asthma, and others.

The inventive liposomes, pharmaceutical agents and/or the kit can be used in the prophylaxis or treatment of pathogenic modifications, preferably (i) inflammations, more preferably (ii) autoimmune diseases, and most preferably (iii) arthritis.

(i) Inflammations in the meaning of the invention are reactions of the organism, mediated by the connective tissue and blood vessels, to an external or internally triggered inflammatory stimulus, with the purpose of eliminating or inactivating the latter and repairing the tissue lesion caused by said stimulus. A triggering effect is caused by mechanical stimuli (foreign bodies, pressure, injury) and other physical factors (ionizing radiation, UV light, heat, cold), chemical substances (alkaline solutions, acids, heavy metals, bacterial toxins, allergens, and immune complexes), and pathogens (microorganisms, worms, insects), or pathologic metabolites, derailed enzymes, malignant tumors. The process begins with a brief arteriolar constriction (as a result of adrenaline effect), with inadequate circulation and tissue alteration, followed by development of classical local inflammatory signs (cardinal symptoms, according to GALEN and CELSUS), i.e., from reddening (=rubor; vascular dilation caused by histamine), heat (=calor; as a result of local increase of metabolism), swelling (=turgor; as a result of secretion of protein-rich liquor from vessel walls changed by histamine, among other things, supported by decelerated blood circulation in the sense of a prestasis up to stasis), pain (=dolor; as a result of increased tissue tension and algogenic inflammation products, e.g. bradykinin), and functional disorders (=functio laesa). The process is accompanied by disorders in the electrolyte metabolism (transmineralization), invasion of neutrophilic granulocytes and monocytes through the vessel walls (cf., leukotaxis), with the purpose of eliminating the inflammatory stimulus and the damaged to necrotic cells (phagocytosis); furthermore, invasion of lymphocyte effector cells, giving rise to formation of specific antibodies against the inflammatory stimulus (immune reaction), and of eosinophiles (during the phase of healing or—at a very early stage—in allergic-hyperergic processes). As a result of the activation of the complement system occurring during the reaction, fragments (C3a and C5a) of this system are liberated which—like histamine and bradykinin—act as inflammation mediators, namely, in the sense of stimulating the chemotaxis of the above-mentioned blood cells; furthermore, the blood coagulation is activated. As a consequence, damage (dystrophia and coagulation necrosis) of the associated organ parenchyma occurs. Depending on the intensity and type of the inflammation, the overall organism responds with fever, stress (cf., adaptation syndrome), leukocytosis and changes in the composition of the plasma proteins (acute-phase reaction), giving rise to an accelerated erythrocyte sedimentation. Preferred inflammations in the meaning of the invention are suppurative, exudative, fibrinous, gangrenescent, granulomatous, hemorrhagic, catarrhal, necrotizing, proliferative or productive, pseudomembranous, serous, specific and/or ulcerous inflammations.

(ii) Autoimmune diseases in the meaning of the invention are diseases entirely or partially due to the formation of autoantibodies and their damaging effect on the overall organism or organ systems, i.e., due to autoaggression. A classification into organ-specific, intermediary and/or systemic autoimmune diseases can be made. Preferred organ-specific autoimmune disease are HASHIMOTO thyroiditis, primary myxedema, thyrotoxicosis (BASEDOW disease), pernicious anemia, ADDISON disease, myasthenia gravis and/or juvenile diabetes mellitus. Preferred intermediary autoimmune diseases are GOODPASTURE syndrome, autoimmune hemolytic anemia, autoimmune leukopenia, idiopathic thrombocytopenia, pemphigus vulgaris, sympathetic ophthalmia, primary bile cirrhosis, autoimmune hepatitis, ulcerative colitis and/or SJÖGREN syndrome. Preferred systemic autoimmune diseases are rheumatoid arthritis, rheumatic fever, systemic lupus erythematosus, dermatomyositis/polymyositis, progressive systemic sclerosis, WEGENER granulomatosis, panarteritis nodosa and/or hypersensitivity angiitis. Typical autoimmune diseases are thyrotoxicosis, thyroid-caused myxedema, HASHIMOTO thyroiditis, generalized endocrinopathy, pernicious anemia, chronic gastritis type A, diseases of single or all corpuscular elements of the blood (for example, autoimmune hemolytic anemia, idiopathic thrombocytopenia or thrombocytopathy; idiopathic leukopenia or agranulocytosis), pemphigus vulgaris and pemphigoid, sympathetic ophthalmia, and numerous forms of uveitis, primarily biliary liver cirrhosis and chronic aggressive autoimmune hepatitis, diabetes mellitus type I, CROHN disease and ulcerative colitis, SJÖGREN syndrome, ADDISON disease, lupus erythematosus disseminatus and discoid form of said disease, as dermatomyositis and scleroderma, rheumatoid arthritis (=primarily chronic polyarthritis), antiglomerular basement membrane nephritis. The basis is an aggressive immune reaction due to breakdown of the immune tolerance to self-determinants and a reduction of the activity of T suppressor cells (with lymphocyte marker T8) or an excess of T helper cells (with lymphocyte marker T4) over the suppressor cells; furthermore, formation of autoantigens is possible e.g. by coupling of host proteins to haptens (e.g. drugs), by ontogenetic tissue not developing until self-tolerance has developed, by protein components demasked as a result of conformational changes of proteins in connection with e.g. infection by viruses or bacteria; and by new proteins formed in association with neoplasias.

(iii) Arthritis in the meaning of the invention is joint inflammation as a generic term for primary or secondary inflammatory joint diseases. Preferred arthritic diseases are juvenile chronic, A. mutilans, paraneoplastic, A. psoriatica, reactive and/or rheumatoid arthritis.

Liposomal formulations of glucocorticoids can also be used with advantage to suppress rejection of grafts or in the treatment of type I diabetes.

A particularly preferred indication is rheumatic arthritis.

Thus, a substantially improved therapeutic effect has been observed with equal amounts of active substance. Antigen-induced arthritis in rats could be suppressed almost completely with 0.375 mg of liposomal dexamethasone phosphate per kg body weight. Animals which received an identical dose of nonencapsulated active substance developed all symptoms of the animal model after short amelioration.

Without intending to be limiting, the implementation of the teaching according to the invention will be illustrated with reference to the following examples.

### EXAMPLE 1

### Preparation of Liposomes filled with Dexamethasone Phosphate

A mixture of 50 mole-% DPPC, 10 mole-% DPPG and 40 mole-% Chol is dissolved in chloroform and subsequently dried completely in vacuum in a rotary evaporator.

The lipid film is added with dexamethasone phosphate solution (25 mg/ml dexamethasone phosphate in 10 mM HEPES, 150 mM NaCl, pH 7.5) in an amount so as to form a 100 mM suspension. Subsequently, this suspension is hydrated in a water bath at 50° C. for 45 minutes with rotating and treated in an ultrasonic bath for another 5 minutes. Thereafter, the solution is frozen.

Following thawing, the liposomes are extruded repeatedly through a membrane having a pore width of 400 nm. Removal of non-entrapped dexamethasone phosphate is effected by means of gel filtration.

### EXAMPLE 2

### Determination of Dexamethasone Phosphate Liberation

Liposomes are prepared as in Example 1 and diluted with buffer (10 mM HEPES, 150 mM NaCl, pH 7.5) to a concentration of 12 mM lipid. Subsequently, they are incubated at 37° C. Aliquots of this incubation solution are taken at well-defined points in time (see Table below). The aliquots taken are measured for their content of dexamethasone phosphate using RP-HPLC.

TABLE 1

| | | | 0 | 24 | 120 |
|---|---|---|---|---|---|
| Formulation | Lipid | mole-% | hours | hours | hours |
| A | DPPC/DPPG/Chol | 50:10:40 | 1.1 | 6.0 | 7.3 |

Liberation of dexamethasone phosphate in % of entrapped amount at different times after incubation at 37° C. in buffer

The liposomes exhibit sufficient stability over the investigated measuring period. Less than 8% of the entrapped active substance is liberated within a period of 5 days.

### EXAMPLE 3

### Use of Liposomal Dexamethasone Phosphate

Antigen-induced arthritis was provoked in test animals (rats) according to Buchner (“Behandlung der antigeninduzierten Arthritis der Ratte mit Anti-Makrophagenprinzipien und monoklonalen Anti-CD4 Antikörpern”, Ph.D. thesis 1996, Friedrich-Alexander University Erlangen-Nuremberg, Germany, p. 129).

Arthritis was provoked on day 0 according to Buchner by intra-articular injection of the antigen into the synovial gap in the right knee. The arthritic animals were treated intravenously with liposomal dexamethasone phosphate (formulation A, 3.75 mg/kg) 6, 24 and 48 hours after induction of arthritis. Free dexamethasone phosphate (formulation K, likewise 3.75 mg/kg) and physiological salt solution (saline) were used as controls. The effect of sample administration was established using the following parameters:
determination of joint swelling (cf., FIG. 1)

histological examination of inflammation parameters and of joint cartilage destruction parameters (cf. FIG. 2 and Table 2)

TABLE 2

| | Degree of cartilage | Degree of joint |
|---|---|---|
| Formulation | destruction | inflammation |
| Saline | 4.0 | 5.5 |
| Formulation K | 2.75 | 3.5 |
| Formulation A | 0.25 | 0.25 |

Degree of joint cartilage destruction and joint inflammation of arthritic animals after treatment

0: no changes compared to healthy knee

6: massive changes compared to healthy knee Both joint cartilage destruction and inflammatory reactions are effectively reduced as a result of treatment with liposomal dexamethasone phosphate. Free active substance at the concentration specified is considerably less effective.

### EXAMPLE 4

### Use of Liposomal Dexamethasone Phosphate in Dose Reduction

Arthritis was provoked in rats in analogy to Example 3, and the rats were treated with the following formulations containing only one tenth of the dexamethasone phosphate dose specified in Example 3:

Formulation A 25 μg=0.375 mg/kg b.w.

Formulation K 25 μg=0.375 mg/kg b.w.

The joint swellings measured are also illustrated in FIG. 1. An almost identical therapeutic effect can be seen with the reduced dose; joint swelling completely disappears after about 3 days with formulation A, which is not the case with formulation K.

### EXAMPLE 5

### Visualization of Liposome Distribution in the Body

The fluorescent dye Cy5.5™ (Amersham Bioscience) was used as label, which dye has an excitation maximum at 675 nm and a fluorescence maximum at 694 nm, i.e., in the near infrared, and can be excited by irradiating through the skin. Cy5.5™-BSA was obtained by coupling Cy5.5™-NHS ester to BSA according to the protocol of Amersham Bioscience.

Liposomes filled with Cy5.5™-BSA, having the composition DPPC/DPPG/Chol 50:10:40, were produced in analogy to Example 1.

Non-entrapped Cy5.5™ was removed by washing (HEPES 10 mM, including 150 mM NaCl) and sedimentation (65,000 rpm with Beckman Rotor 100.4, 40 min, at 21° C.), and the liposomes were diluted with physiological saline to 6.5 mM in lipid (Cy5.5™ content: 8 nmol/ml).

Test animals: mice with established AIA (antigen-induced arthritis), in analogy as described in the previous example.

On the 7^{th} day after provoking arthritis, the animals received an intravenous single application of 100 μl of liposome-encapsulated Cy5.5™ into their tail vein.

Fluorescence measurement was effected after 6 hours using NIR photography (near infrared), wherein arthritic and healthy knee joints were measured separately.

FIG. 3 shows sites, as bright areas, where accumulation of the Cy5.5™ dye has taken place. Approximately, the signal intensities are as follows:

900 in arthritic joint;

600 in healthy joint;

2500 in liver and spleen.

By measuring the increase of the fluorescence signal, it was possible to monitor the liposome uptake into the joints in a time series.

This is illustrated in FIG. 4. As can be seen, the arthritic joint has higher accumulation of Cy5.5™ than the healthy joint over a period spanning the first 12 to 24 hours.

### EXAMPLE 6

### Preparation of Amphoteric Liposomes filled with Dexamethasone Phosphate

A mixture of 60 mole.-% POPC, 20 mole-% MoChol and 20 mole-% CHEMS is dissolved in chloroform and subsequently dried completely in vacuum in a rotary evaporator.

The lipid film is added with dexamethasone phosphate solution (25 mg/ml dexamethasone phosphate in 10 mM HEPES, 150 mM NaCl, pH 7.5) in an amount so as to form a 100 mM suspension. Subsequently, this suspension is hydrated in a water bath at 50° C. for 45 minutes with rotating and treated in an ultrasonic bath for another 5 minutes. Thereafter, the solution is frozen.

Following thawing, the liposomes are extruded repeatedly through a membrane having a pore width of 400 nm. Removal of non-entrapped dexamethasone phosphate is effected by means of gel filtration.

In analogy, the following liposomes filled with dexamethasone phosphate are produced:

TABLE 3

| Lipid | mole-% |
|---|---|
| POPC/MoChol/CHEMS | 60:20:20 |
| POPC/HisChol/CHEMS | 60:20:20 |
| POPC/DOTAP/CHEMS | 60:10:30 |
| POPC/HistChol/Chol | 60:20:20 |
| DPPC/AC/Chol | 50:10:40 |

Examples of possible liposome formulations for inclusion of water-soluble glucocorticoids

### EXAMPLE 7

### Determination of Dexamethasone Phosphate Liberation from Amphoteric Liposomes

Liposomes are prepared as in Example 6 and diluted with buffer (10 mM HEPES, 150 mM NaCl, pH 7.5) to a concentration of 12 mM lipid. Subsequently, they are incubated at 37° C. Aliquots of this incubation solution are taken at well-defined points in time (see Table below). The aliquots taken are measured for their content of dexamethasone phosphate using RP-HPLC.

TABLE 4

| | | | 0 | 24 | 120 |
|---|---|---|---|---|---|
| Formulation | Lipid | mole-% | hours | hours | hours |
| | POPC/MoChol/CHEMS | 60:20:20 | 4.6 | 10.5 | 10.0 |
| B | POPC/HisChol/CHEMS | 60:20:20 | 6.7 | 14.1 | 10.8 |
| C | POPC/DOTAP/CHEMS | 60:10:30 | 4.4 | 4.2 | — |
| | POPC/HistChol/Chol | 60:20:20 | 8.7 | 7.0 | — |
| | DPPC/AC/Chol | 50:10:40 | 1.7 | 7.8 | 7.4 |

Liberation of dexamethasone phosphate in % of entrapped amount at different times after incubation at 37° C. in buffer

The different liposomes exhibit sufficient stability over the investigated measuring period. Less than 6%, frequently less than 4% of the entrapped active substance is liberated within a period of 5 days.

### EXAMPLE 8

### Use of Dexamethasone Phosphate formulated in Amphoteric Liposomes

In analogy to Example 3, antigen-induced arthritis was provoked in the test animals. Arthritis was provoked on day 0 by intra-articular injection of the antigen into the synovial gap in the right knee. The arthritic animals were treated intravenously with liposomal dexamethasone phosphate (formulation B, C, 3.75 mg/kg each time, according to Table 4) 6, 24 and 48 hours after induction of arthritis. Free dexamethasone phosphate (formulation K, likewise 3.75 mg/kg) and physiological salt solution (saline) were used as controls. The effect of sample administration was established using the following parameters:
determination of joint swelling (cf., FIG. 5)

histological examination of inflammation parameters and of joint cartilage destruction parameters (cf., FIG. 2 and Table 5)

TABLE 5

| | Degree of cartilage | Degree of joint |
|---|---|---|
| Formulation | destruction | inflammation |
| Saline | 4.0 | 5.5 |
| Formulation K | 2.75 | 3.5 |
| Formulation B | 0 | 0.25 |
| Formulation C | 1.0 | 1.5 |

Degree of joint cartilage destruction and joint inflammation of arthritic animals after treatment

0: no changes compared to healthy knee

6: massive changes compared to healthy knee

Both joint cartilage destruction and inflammatory reactions are effectively reduced as a result of treatment with liposomal dexamethasone phosphate. Free active substance at the concentration specified is considerably less effective.

### EXAMPLE 9

### Dose Reduction

Arthritis was provoked in rats in analogy to Example 8, and the rats were treated with the following formulations containing only one tenth of the dexamethasone phosphate dose specified in Example 8:

Formulation A 25 μg=0.375 mg/kg b.w.

Formulation K 25 μg=0.375 mg/kg b.w.

The joint swellings measured are also illustrated in FIG. 5. An almost identical therapeutic effect can be seen with the reduced dose; joint swelling completely disappears after about 3 days with formulation B, which is not the case with formulation K.

### DESCRIPTION OF THE FIGURES

FIG. 1 Joint swelling in arthritic animals over the test period of 21 days after provoking arthritis in the right knee in comparison to the healthy left knee.

FIG. 2 Histological sections of arthritic knee joints. Hematoxylin/eosin staining, magnified 92fold
K—formulation K, massive inflammation and destruction of cartilage and bone
A—formulation A, slight inflammatory infiltration
C—formulation C, no inflammatory reaction

FIG. 3 Infrared fluorescence of a rat treated with Cy5.5™-BSA-filled liposomes of formulation DPPC/DPPG/Chol 50:10:40.

FIG. 4 Comparison of time-dependent liposome accumulation in arthritic versus non-arthritic joint.

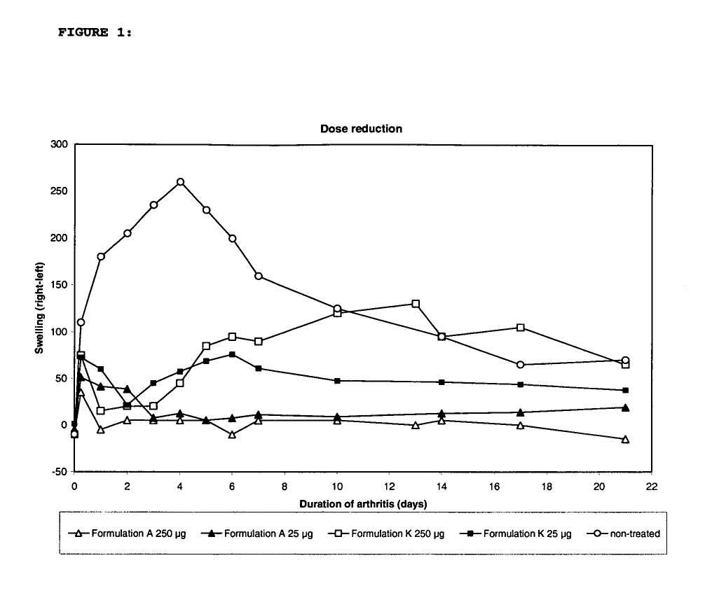

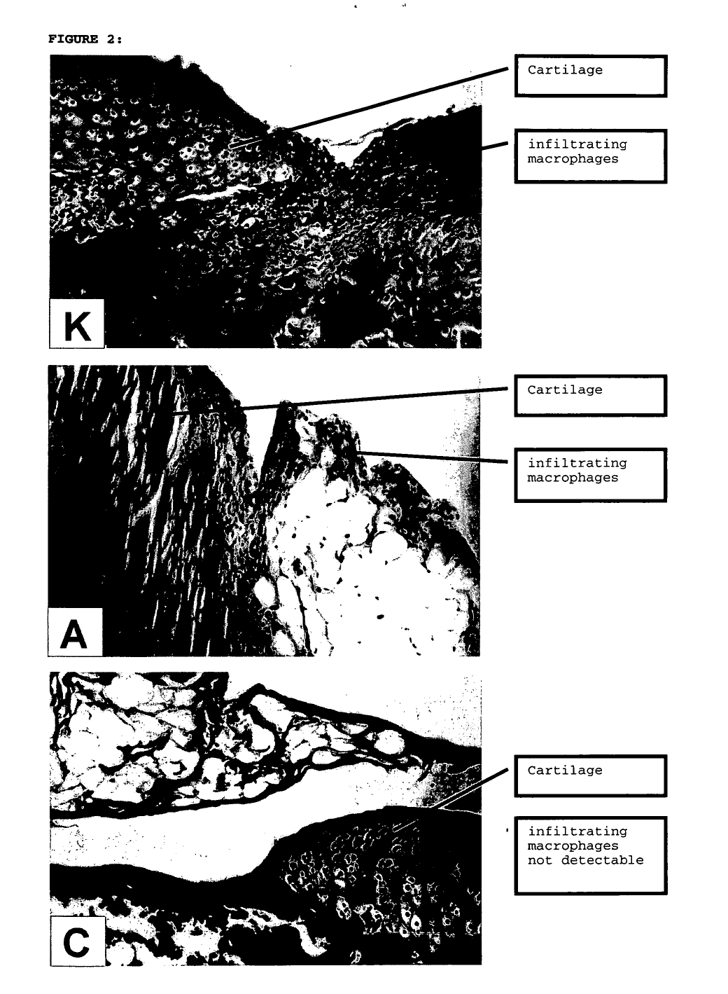

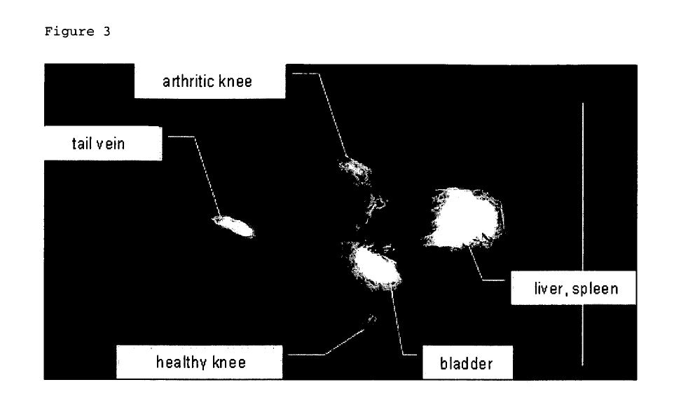

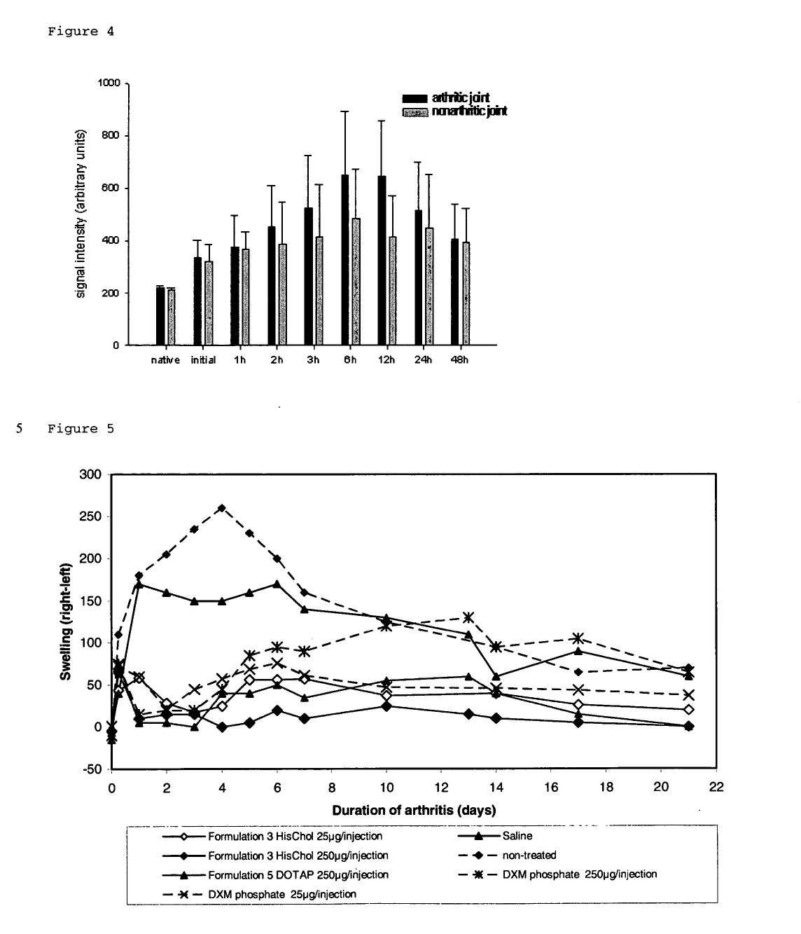

FIG. 5 Joint swell in arthritic animals over the test period of 21 days after provoking arthritis in the right knee in comparison to the healthy left knee.
Formulation 3 HisChol=formulation B
Formulation 5 DOTAP=formulation C
Formulation DXM phosphate=formulation K

Abbreviations:
HistChol Na-Histidinylcholesterol hemisuccinate
DOTAP N-[1-(2,3-Dioleoyloxy)propyl]-N,N,N-trimethylammonium
MoChol 4-(2-Aminoethyl)morpholinocholesterol hemisuccinate
HisChol Histaminylcholesterol hemisuccinate
AC Palmitoylcarnosine (acylcarnosine)
CHEMS Cholesterol hemisuccinate
DPPC Dipalmitoylphosphatidyl choline
DPPG Dipalmitoylphosphatidyl glycerol
POPC Palmitoyloleoylphosphatidyl choline
Chol Cholesterol
BSA Bovine serum albumin

Literature

Mizushima, Y.; Hamano, T.; Yokoyama, K. (1982): Tissue distribution and anti-inflammatory activity of corticosteroids incorporated in lipid emulsion. Ann. Rheum. Dis. 41(3), 263-267

Yokoyama, K.; Okamoto, H.; Watanabe, M.; Suyama, T.; Mizushima, Y. (1985), Development of a corticosteroid incorporated in lipid microspheres (liposteroid). Drugs Exp. Clin. Res. 11(9), 611-620

Bonanomi, M. H.; Velvart, M.; Weder, H. G. (1987), Fate of different kinds of liposomes containing dexamethasone palmitate after intra-articular injection into rabbit joints. J. Microencapsul. 4(3), 189-200

Loftsson, T. & Stefansson, E. (2002): Cyclodextrins in eye drop formulations: enhanced topical delivery of corticosteroids to the eye. Acta Ophthalmol. Scand. 80(2), 144-150

Yano, H.; Hirayama, F.; Kamada, M.; Arima, H.; Uekama, K. (2002), Colon-specific delivery of prednisolone-appended alphacyclodextrin conjugate: alleviation of systemic side effect after oral administration. J. Control Release 79(1-3), 103-112

Storm, G. & Richmond, P. L. (2001), Pegylated liposomal doxorubicin: tolerability and toxicity. Pharmacotherapy 21(6), 751-763

Metselaar, J. M.; Wauben, M. H. M.; Boerman, O. C.; van Lent, P. L.; Storm, G. (2002), Long-circulating liposomes for i.v. targeted delivery of glucocorticoids in arthritis. Cell Mol. Biol. Lett. 7(2), 291-292

Hussein, M. A.; Wood, L.; His, E.; Srkalovic, G.; Karam, M.; Elson, P.; Bukowski, R. M. (2002), A phase II trial of pegylated liposomal doxorubicin, vincristine, and reduced-dose dexamathasone combination therapy in newly diagnosed multiple myeloma patients. Cancer 95(10), 2160-2168

Dams, E. T. M.; Laverman, P.; Oyen, W. J. G.; Storm, G.; Scherphof, G.; van der Meer, J. W. M.; Corstens, F. H. M.; Boerman, O. C. (2000), Accelerated blood clearance and altered biodistribution of repeated injections of sterically stabilized liposomes. The Journal of Pharmacology and Experimental Therapeutics, 292(3), 1071-1079

EP 1 046 394 (Terumo Corp.)

WO 02/45688 (Yamanouchi Euro BV), Parenteral compositions for site-specific treatment of inflammatory disorders comprise liposomes composed of non-charged vesicle-forming lipids and a water-soluble corticosteroid.

WO 95/15746 (Univ. London School Pharmacy), New liposomes, esp. for drug delivery—having an internal aq. phase cont. complex of active cpd. with receptor, e.g. for rendering hydrophobic drugs hydrophilic.

WO 94/07466 (Liposome Technology Inc.), Treatment of inflamed tissue—using liposomes contg. a lipid derivatised with polyethylene glycol entrapping a therapeutic cpd.

DE 27 12 030 (Imperial Chem Ind., 1977), Antiinflammatory medicaments based on liposomes—contg. a lipophilically subst. steroid

1-14. (canceled)

15. A liposomal formulation, wherein water-soluble glucocorticoids are present in the interior aqueous phase of the liposomes, and wherein the liposomes are characterized by having a size between 150 and 500 nm, exhibiting an excess of negative charge at a physiological pH of 7.5 and having a cholesterol content between 35 and 50 mole-%, and wherein the liposomes do not comprise any amphiphilic-modified polyethylene glycol.

16. The liposomal formulation according to claim 15, wherein
the cholesterol content is between 35 and 45 mole-%.

17. The liposomal formulation according to claim 15, wherein
the neutral lipids are selected from the group of dimyristoylphosphatidyl choline, dipalmitoylphosphatidyl choline, palmitoyloleoylphosphatidyl choline, distearoyl-phosphatidyl choline and/or cholesterol, or comprise purified choline fractions of natural origin, such as soy phosphatidyl choline or egg phosphatidyl choline.

18. The liposomal formulation according to claim 15, wherein
the negative charge of the liposomal membrane is provided by anionic lipids alone, said anionic lipids comprising phosphatidyl glycerol and phosphatidyl serine, more preferably phosphatidyl glycerol, especially preferably dipalmitoyl phosphatidyl glycerol, and that the molar proportion of said lipids is no higher than 20 mole-%, more preferably between 5 and 15 mole-%.

19. The liposomal formulation according to claim 15, wherein
the liposomes are amphoteric liposomes and have an excess of positive charge at pH 5.

20. The liposomal formulation according to claim 18, wherein
the liposome membrane contains neutral, cationic and anionic lipids, and that the molar proportion of anionic lipids is no higher than 40 mole-%, more preferably 5 to 30 mole-%, that of cationic lipids no higher than 40 mole-%, more preferably 5 to 30 mole-%, and that of neutral lipids 30 to 80 mole-%, more preferably 40 to 70 mole-%.

21. The liposomal formulation according to claim 20, wherein
the liposome membrane contains neutral and amphoteric lipids, and that the molar proportion of amphoteric lipids comprises 5 to 40 mole-%, and that of neutral lipids between 30 and 80 mole-%, more preferably between 40 and 70 mole-%.

22. The liposomal formulation according to claim 15, wherein
the water-soluble glucocorticoids are phosphate esters, glycosides or sulfate esters thereof.

23. The liposomal formulation according to claim 22, wherein
the water-soluble glucocorticoid is selected from the group of dexamethasone phosphate, dexamethasone dihydrogen phosphate disodium, triamcinolone acetonide phosphate, prednisolone phosphate.

24. A method for treatment of a human or animal in need thereof, the method comprising administering to said human or animal a liposomal formulation liposomal formulation, wherein water-soluble glucocorticoids are present in the interior aqueous phase of the liposomes, and wherein the liposomes are characterized by having a size between 150 and 500 nm, exhibiting an excess of negative charge at a physiological pH of 7.5 and having a cholesterol content between 35 and 50 mole-%, and wherein the liposomes do not comprise any amphiphilic-modified polyethylene glycol in systemic application.

25. The method as in claim 24, wherein said treatment is for an inflammatory disease.

26. A method for therapeutic treatment of rheumatic arthritis, comprising systemic application in a human or animal a rheumatic arthritis liposomal formulation, wherein
water-soluble glucocorticoids are present in the interior aqueous phase of the liposomes, and wherein the liposomes do not comprise any amphiphilic-modified polymers such as polyethylene glycol-phosphatidyl ethanolamine.

27. A method for the therapy of inflammatory disease in a human or animal, comprising systemic application to a human or animal a liposomal formulation, wherein water-soluble glucocorticoids are present in the interior aqueous phase of the liposomes, and wherein the liposomes are characterized by having a size between 150 and 500 nm, exhibiting an excess of negative charge at a physiological pH of 7.5 and having a cholesterol content between 35 and 50 mole-%, and wherein the liposomes do not comprise any amphiphilic-modified polyethylene glycol.

28. A method for the therapy of rheumatic arthritis in a human or animal, comprising systemic application to a human or animal a liposomal formulation, wherein water-soluble glucocorticoids are present in the interior aqueous phase of the liposomes, and wherein the liposomes are characterized by having a size between 150 and 500 nm, exhibiting an excess of negative charge at a physiological pH of 7.5 and having a cholesterol content between 35 and 50 mole-%, and wherein the liposomes do not comprise any amphiphilic-modified polyethylene glycol
